(19) United States
(12) Patent Application Publication
(10) Pub. No.: US 2010/0272652 A1
(43) Pub. Date: Oct. 28, 2010

(54) DETERMINATION OF TRACERS WITHIN SUBJECTS

(75) Inventor: Douglas A. Levinson, Sherborn, MA (US)

Correspondence Address: WOLF GREENFIELD & SACKS, P.C., 600 ATLANTIC AVENUE, BOSTON, MA, 02210-2206, US

(73) Assignee: Seventh Sense Biosystems, Inc., Cambridge, MA (US)

(21) Appl. No.: 12/748,316

(22) Filed: Mar. 26, 2010

Related U.S. Application Data
(60) Provisional application No. 61/163,733, filed on Mar. 26, 2009.

(51) Int. Cl. A61B 5/00; A61P 43/00

(52) U.S. Cl. 424/9.8

(57) ABSTRACT

The present invention generally relates to the use of tracers, for example, to track a substance that has been administered to a subject. A tracer may be separate from the substance to be administered, or in some cases, the tracer and the substance are chemically bonded to each other. When administered to a subject (e.g., ingested), the tracer and the substance to be administered may be absorbed and/or metabolized by the subject, and the pharmacokinetic activity of the tracer within the subject may be related to the pharmacokinetic activity of the substance. Thus, by following the pharmacokinetic activity of the tracer, the pharmacokinetic activity of the substance may be determined. The tracer itself may be determined within a subject using any suitable method, depending on the tracer. As an example, the tracer may be determined by administering an indicator able to interact with the tracer. For instance, the indicator may be applied to the skin of the subject, and the indicator may give a different visual appearance based on the concentration of the tracer, or otherwise exhibit a determinable change in a property of the indicator. Other aspects of the invention are generally directed to methods of making or using tracers, methods of promoting the making or use of such tracers, kits involving such tracers, and the like.

### RELATED APPLICATIONS

This application claims the benefit of U.S. Provisional Patent Application Ser. No. 61/163,733, filed Mar. 26, 2009, entitled “Determination of Tracers within Subjects,” by Levinson, incorporated herein by reference.

### FIELD OF INVENTION

The present invention generally relates to the use of tracers, for example, to track a substance that has been administered to a subject.

### BACKGROUND

It is often desirable to follow the course of a drug or other therapeutic agent that has been administered to a subject. For example, the pharmacokinetics of absorption of the drug may differ between different subjects, or it may be desirable to follow the concentration of the drug within the subject. Many drugs have a relatively narrow “therapeutic window” in which they are effective: below a certain concentration, the drug may be ineffective for its intended use, but above a certain concentration, the drug is toxic to the subject. In addition, this therapeutic window may differ for different subjects, e.g., due to differences in physiology between subjects.

In many cases, it is difficult to follow the course of a drug or other therapeutic agent that has been administered to a subject. Drugs are often present or effective within the body at relatively low concentrations, and it is difficult to repeatedly sample the bloodstream of a subject in order to determine drug absorption pharmacokinetics. Thus, improvements in the ability to follow the course of a drug or other therapeutic agent are needed.

### SUMMARY OF THE INVENTION

The present invention generally relates to the use of tracers, for example, to track a substance that has been administered to a subject. The subject matter of the present invention involves, in some cases, interrelated products, alternative solutions to a particular problem, and/or a plurality of different uses of one or more systems and/or articles.

In one aspect, the present invention is directed to a method. In a first set of embodiments, the method includes acts of offering, to a subject, a composition comprising a tracer and a substance able to be introduced and be determined in the subject, and determining whether the composition was administered to the subject by determining the tracer within the subject. The method, in another set of embodiments, includes acts of administering a composition comprising a tracer and a substance to a subject, and determining activity of the substance within the subject by determining the tracer within the subject.

The method, in yet another set of embodiments, includes acts of administering a composition comprising a tracer and a pharmaceutically acceptable carrier to a subject, and administering an indicator responsive to the tracer to the skin of the subject. In still another set of embodiments, the method includes acts of administering, to a subject, a composition comprising a tracer and a substance able to be introduced and be determined in the subject, and administering an indicator responsive to the tracer to the skin of the subject, the indicator able to change a property upon response to the tracer.

In one set of embodiments, the method includes an act of offering, to a subject, an ingestible composition comprising a tracer and an ingestible substance, and a dermally-applicable composition comprising an indicator able to change a visual property upon exposure to the tracer.

The method, in another set of embodiments, includes acts of determining a substance within a subject by monitoring a tracer within the subject. In some cases, the tracer and the substance are chemically unbonded to each other.

According to one set of embodiments, the method includes an act of determining an exogenous tracer within a subject indicative of a substance administered to the subject.

In another set of embodiments, the method includes acts of providing a substance, the presence and/or amount of which is to be determined in association with a subject; offering, to the subject, a composition comprising the substance and a tracer able to be introduced into and be determined in association with the subject; and determining whether the substance was administered to the subject by determining the tracer in association with the subject.

The method, according to yet another set of embodiments, includes an act of offering, to a subject, an ingestible composition comprising a tracer and an ingestible substance, and a device comprising an indicator able to change a visual property upon exposure to the tracer.

Another aspect of the present invention is directed to a kit. The kit, in one set of embodiments, includes a composition comprising a tracer and a carrier containing the tracer, and a skin delivery device comprising an indicator responsive to the tracer. In another set of embodiments, the kit includes a composition comprising a tracer and a substance, and a skin delivery device comprising an indicator responsive to the tracer. In yet another set of embodiments, the kit includes a tracer, a substance; and optionally, instructions for administering the tracer and the substance to a subject, and/or determining the tracer within the subject.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C illustrate various embodiments in which a tracer can be used to determine a substance delivered to a subject.

### DETAILED DESCRIPTION

The present invention generally relates to the use of tracers, for example, to track a substance that has been administered to a subject. A tracer may be separate from the substance to be administered, or in some cases, the tracer and the substance are chemically bonded to each other. When administered to a subject (e.g., ingested), the tracer and the substance to be administered may be absorbed and/or metabolized by the subject, and the pharmacokinetic activity of the tracer within the subject may be related to the pharmacokinetic activity of the substance. Thus, by following the pharmacokinetic activity of the tracer, the pharmacokinetic activity of the substance may be determined. The tracer itself may be determined within a subject using any suitable method, depending on the tracer. As an example, the tracer may be determined by administering an indicator able to interact with the tracer to the subject. For instance, the indicator may be applied to the skin of the subject, and the indicator may give a different visual appearance based on the concentration of the tracer, or otherwise exhibit a determinable change in a property of the indicator. Other aspects of the invention are generally directed to methods of making or using tracers, methods of promoting the making or use of such tracers, kits involving such tracers, and the like.

As used herein, a “tracer” is a species that can be determined within a subject, typically upon interaction with an indicator. The tracer may be determined in the skin of the subject, or a bodily fluid such as blood or interstitial fluid may be withdrawn from a subject and the tracer determined within the withdrawn fluid, thereby indicating the presence and/or amount of tracer within the subject. Thus, in one set of embodiments, a tracer may be determined in association with the subject, i.e., the tracer may be determined while the tracer is physically within the subject, e.g., within the skin of the subject, and/or the tracer may be determined after being removed from the subject in some fashion, e.g., by being withdrawn within a bodily fluid such as blood or interstitial fluid. The tracer is typically, but need not be, an auxiliary species administered along with the substance, the presence and/or quantity of which is to be determined in association with the subject, and in many cases the tracer has no purpose in relation to the subject other than its function as a tracer.

An “indicator” is a species that exhibits a change in a determinable property upon interaction with a tracer. However, it should be understood that an indicator is not necessarily required in all embodiments of the invention. In some cases, the tracer itself is determinable in some fashion. For example, the tracer may be radioactive or fluorescent in some cases, although in other cases, the tracer may not be radioactive and/or fluorescent. The determinable change in the tracer and/or the indicator may be a visual change such as a change in appearance (e.g., color), a change in temperature, a change in sensation, or the like. The tracer itself may be any suitable compound that can be administered to the subject. In some cases, the determinable change may be one that can be determined by a human without the use of any equipment, for example, visually, tactilely, or the like. In other cases, however, the determinable change may be determinable using suitable instrumentation.

In some cases, the tracer is chosen to have relatively little, or essentially no, biological activity, and can be determined mainly by its interaction with the indicator. However, in other cases, the tracer may have some biological activity. For instance, the amount of biological activity of the tracer within the subject may be predictable. As an example, a tracer may be cleared by the kidneys from the bloodstream at a certain rate, and by determining the concentration of tracer within the subject, e.g., by determining a change in a determinable property in an indicator, and correcting for the clearance rate of the tracer, the pharmacokinetic activity of the tracer within the subject may be determined, and used to determine the pharmacokinetic activity of a substance administered to the subject. Usually, the tracer is produced externally or exogenously, then administered to the subject as discussed below. Non-limiting examples of tracers include certain proteins or carbohydrates such as inulin, or small molecules (typically less than about 1000 Da) such as creatinine. The tracer may be relatively non-toxic in some cases. In one set of embodiments, the tracer is a molecule that has a relatively high rate of clearance from the body. For instance, the half-life of the tracer within the body may be less than about 3 days, less than about 2 days, less than about 1 day, less than about 18 hours, less than about 12 hours, less than about 9 hours, less than about 3 hours, or less than about 1 hour. In some cases, the tracer may include poly(ethylene) glycol, for example, PEG 300, PEG 400, PEG 2000, PEG 3350, or PEG 8000 (where “PEG” stands for poly(ethylene) glycol and the number indicates the molecular weight).

In one set of embodiments, a tracer is administered to a subject along with a substance, such as a drug or other therapeutic agent, and by determining the tracer within the subject, the substance may also be determined within the subject, either directly, or by utilizing various corrections or correlations. Thus, the tracer may be a separate entity from the substance, at least in certain cases. The subject is usually human, although non-human subjects may be used in certain instances, for example, other mammals such as a dog, a cat, a horse, a rabbit, a cow, a pig, a sheep, a goat, a rat (e.g., Rattus Norvegicus), a mouse (e.g., Mus musculus), a guinea pig, a hamster, a primate (e.g., a monkey, a chimpanzee, a baboon, an ape, a gorilla, etc.), or the like.

“Determine,” in this context, generally refers to the analysis of a species, for example, quantitatively or qualitatively, and/or the detection of the presence or absence of the species. For example, a tracer may be determined in a subject qualitatively (e.g., whether the tracer is present, and therefore whether subject has taken the substance or not), and/or quantitatively (e.g., determining how much of the tracer is present, and therefore how much of the substance the subject has taken). “Determining” may also refer to the analysis of an interaction between two or more species, for example, quantitatively or qualitatively, and/or by detecting the presence or absence of the interaction, e.g. determination of the binding between two species. “Determining” also means detecting or quantifying interaction between species.

As a non-limiting example, the tracer may exhibit substantially the same pharmacokinetic activity as the substance, or at least exhibit certain pharmacokinetic activities indicative of the substance. For instance, the tracer may exhibit similar absorption and/or distribution rates within the body, the same duration within the body, the same metabolism within the body, or the same excretion rates from the body, e.g., through the urine. In other cases, however, the tracer and the substance may exhibit substantially different pharmacokinetic parameters. For example, the tracer may exhibit substantially slower or faster absorption or distribution within the body. However, by determining the tracer, e.g., using an indicator, an estimate of the pharmacokinetic activity of the substance within the body may still be obtained. In one embodiment, it may be sufficient to simply determine whether the tracer is present or absent in the body, and then infer that the substance is also present or absent in the body based on the tracer (for example, if the subject is given a composition that comprises both the tracer and the substance to be administered as a single entity). In some cases, the amount of tracer delivered to the subject may also be controlled in some fashion, for example, such that the certain pharmacokinetic activities of the tracer are substantially similar to the pharmacokinetic activities of the substance also administered to the subject. The substance may be any substance to be delivered to a subject, in which a determination of the substance within the subject is desired. As non-limiting examples, the substance may be an alcoholic beverage or a drug that is administered with a tracer, and the indicator used to determine whether the subject has indeed taken the substance or not. For example, the subject may be one who has trouble with memory; by visually determining an indicator (e.g., in the skin), whether the tracer (and thus, the substance) has been administered (or self-administered) to the subject may be determined.

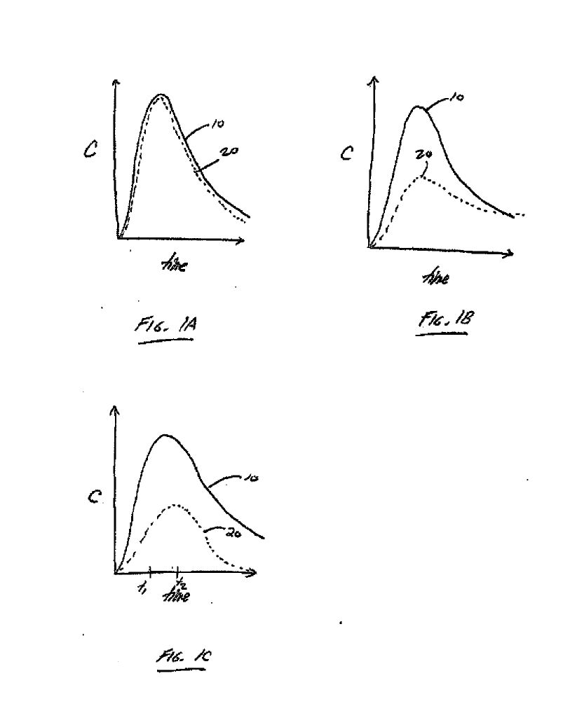

Non-limiting examples of such profiles are now discussed relative to FIG. 1. In FIG. 1A, a plot of concentration versus time can be seen for both a tracer and a substance delivered to a subject. Although the concentration of the tracer 10 may be readily determinable with respect to time in a subject (e.g., as discussed herein), the concentration of the substance 20 itself may be difficult to determine in practice; thus, by determining the tracer within the subject, information regarding the substance may also inferred. Thus, FIG. 1A shows an example where the pharmacokinetic activity of the substance within the body and the tracer are substantially the same. By determining the pharmacokinetic activity of the tracer, the pharmacokinetic activity of the substance within the body can also be determined. FIG. 1B shows a similar example, where the pharmacokinetic activities of the tracer and the substance within the body are not identical. However, information regarding the pharmacokinetic activity of the tracer may still be useful in determining the pharmacokinetic activity of the substance, since both have similar peaks in concentration, although the actual concentration profiles may differ. FIG. 1C illustrates another example, where the peak in time for the tracer (t₁) can be used to determine the peak in time (t₂) for the activity of the substance delivered to the body.

The tracer may be administered to the subject using any suitable method. For example, the tracer may be administered orally, vaginally, rectally, buccally, pulmonary, topically, nasally, transdermally, through parenteral injection or implantation, via surgical administration, or any other suitable method of administration. The tracer may be delivered systemically, or in some cases locally, e.g., at a site proximate an indicator. The tracer may also be administered by the subject (i.e., self-administered), or offered and/or administered to the subject by someone else, e.g., a doctor or a nurse. In addition, techniques discussed below that may be useful for delivering an indicator to a subject may also be useful for delivering a tracer to the subject. As discussed, the tracer and the indicator need not be delivered using the same route of administration (although they can be), and they also need not be delivered simultaneously. For example, the indicator may be rubbed onto the surface of the skin or injected into the skin, while the tracer may be delivered orally, or injected into the bloodstream of the subject.

The tracer may interact with an indicator within the subject. As mentioned, an indicator is a species that can interact with the tracer and exhibit a change in a determinable property upon such an interaction. For instance, the indicator may change appearance or colors in the presence or in the absence of the tracer, e.g., the indicator may exhibit a first color at a first concentration of the tracer and a second color at a second concentration of the tracer, or the tracer may exhibit a range of colors depending on the concentration of the tracer. The indicator may, in certain cases, be immobilized within the subject, e.g., within a depot in the skin. For instance, the indicator may be immobilized such that at least about 90% or at least about 95% of the indicator administered to the subject stays in the location in which it was administered. In some cases, the change can be determined by a human without the use of any equipment. Non-limiting examples include changes in appearance (e.g., color), temperature changes, chemical reactions (e.g., capsaicin) which can be sensed by the subject (e.g., as a feeling of pain), or the like. Examples of capsaicin and capsaicin-like molecules include, but are not limited to, dihydrocapsaicin, nordihydrocapsaicin, homodihydrocapsaicin, homocapsaicin, or nonivamide.

As additional examples, the indicator may include antibodies, enzymes, indicator dyes, or the like which are able to interact with a tracer, and which may exhibit a change in a determinable property, such as a change in color or aggregation, upon such an interaction. As a non-limiting example, in one embodiment, an indicator comprising an antibody may bind to a tracer (e.g., inulin), and upon binding, aggregation of antibodies (e.g., multiple antibodies to the same target, primary antibodies and secondary antibodies where the secondary antibody is labeled, etc.) may be used to determine the tracer. Those of ordinary skill in the art will know of techniques for raising indicators such as antibodies against a specific target.

In certain embodiments, the indicator may comprise particles such as microparticles or nanoparticles, and in some cases, the particles may be anisotropic particles. Specific examples of such particles are discussed in detail below. In some cases, a plurality of particles may be used, and in some cases, some, or substantially all, of the particles may be the same. For example, at least about 10%, at least about 30%, at least about 40%, at least about 50%, at least about 60%, at least about 70%, at least about 80%, at least about 90%, at least about 95%, or at least about 99% of the particles may have the same shape, and/or may have the same composition. For example, in one embodiment, at least about 50% of the particles may be anisotropic. In certain instances, the particles may contain a binding partner on the surface and/or within the particle. Thus, for example, the tracer may interact with the indicator by binding to a binding partner, which may cause a change in a property of the indicator, such as a change in color, aggregation, temperature, or the like.

As an example, the indicator may comprise reactants that, when brought together, are able to cause a reaction. For instance, the first and second reactants can produce heat (e.g., as in an exothermic reaction), cold (e.g., as in an endothermic reaction), a change in color, a product which can then be determined, or the like. As another example, a reaction between the first and second reactants may cause the release of a material. In some cases, the material may be one that can be sensed by a subject, e.g., capsaicin, an acid, an allergen, or the like. Accordingly, the subject may sense the change as a change in temperature, pain, itchiness, swelling, or the like. Thus, for instance, a tracer may cause aggregation of an indicator to occur, thereby brining together various reactants that can react. For instance, the indicator may contain particles having one or more binding partners able to recognize the tracer, and interaction of the tracer with the indicator may cause aggregation of the indicator to occur, which may be determined visually, tactilely, or the like.

The term “binding partner” refers to a molecule that can undergo binding with a particular molecule, e.g., a tracer. For example, the binding may be highly specific and/or non-covalent. Binding partners which form highly specific, non-covalent, physiochemical interactions with one another are defined herein as “complementary.” Biological binding partners are examples. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. For example, Protein A is usually regarded as a “non-specific” or semi-specific binder. As another example, the particles may contain an enzyme such as glucose oxidase or glucose 1-dehydrogenase, or a lectin such as concanavalin A that is able to bind to glucose.

As additional examples, binding partners may include antibody/antigen pairs, ligand/receptor pairs, enzyme/substrate pairs and complementary nucleic acids or aptamers. Examples of suitable epitopes which may be used for antibody/antigen binding pairs include, but are not limited to, HA, FLAG, c-Myc, glutatione-S-transferase, His₆, GFP, DIG, biotin and avidin. Antibodies may be monoclonal or polyclonal. Suitable antibodies for use as binding partners include antigen-binding fragments, including separate heavy chains, light chains Fab, Fab′, F(ab′)₂, Fabc, and Fv. Antibodies also include bispecific or bifunctional antibodies. Exemplary binding partners include biotin/avidin, biotin/streptavidin, biotin/neutravidin and glutathione-S-transferase/glutathione.

The term “binding” generally refers to the interaction between a corresponding pair of molecules or surfaces that exhibit mutual affinity or binding capacity, typically due to specific or non-specific binding or interaction, including, but not limited to, biochemical, physiological, and/or chemical interactions. The binding may be between biological molecules, including proteins, nucleic acids, glycoproteins, carbohydrates, hormones, or the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc. As another example, the binding agent may be a chelating agent (e.g., ethylenediaminetetraacetic acid) or an ion selective polymer (e.g., a block copolymer such as poly(carbonate-b-dimethylsiloxane), a crown ether, or the like). As another example, the binding partners may be biotin and streptavidin, or the binding partners may be various antibodies raised against a protein.

The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair, the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding may be by one or more of a variety of mechanisms including, but not limited to ionic interactions or electrostatic interactions, covalent interactions, hydrophobic interactions, van der Waals interactions, etc.

The indicator may be administered to the subject using any suitable technique, depending on the nature of the indicator. Examples include, but are not limited to, orally, vaginally, rectally, buccally, pulmonary, topically, nasally, transdermally, through parenteral injection or implantation, via surgical administration, or any other suitable method of administration. The method of administering the indicator to the subject need not be the same as the method of administering the tracer to the subject. The indicator may also be administered by the subject (i.e., self-administered), or offered and/or administered to the subject by someone else, e.g., a doctor or a nurse. In addition, the indicator may be determined using any suitable technique. As discussed, in one set of embodiments, the indicator may be determined without the use of any equipment, e.g., determined visually, tactilely, or the like. However, in other embodiments, various sensors may be used to determine the indicator, including, but not limited to, pressure or temperature measurements, spectroscopy such as infrared, absorption, fluorescence, UV/visible, FTIR (“Fourier Transform Infrared Spectroscopy”), or Raman; piezoelectric measurements; immunoassays; electrochemical measurements; optical measurements such as optical density measurements; circular dichroism; or the like.

In certain cases, the indicator is delivered to the skin of the subject. Any suitable skin delivery device may be used to deliver the indicator, for example, using a needle such as a hypodermic needle or microneedles, a patch, or jet injectors such as those discussed below. Hypodermic needles are well-known to those of ordinary skill in the art, and can be obtained commercially with a range of needle gauges. For example, the needle may be in the 20-30 gauge range, or the needle may be 32 gauge, 33 gauge, 34 gauge, etc. As another example, microneedles such as those disclosed in U.S. Pat. No. 6,334,856, issued Jan. 1, 2002, entitled “Microneedle Devices and Methods of Manufacture and Use Thereof,” by Allen, et al., may be used to deliver materials to the skin. The microneedles may be formed from any suitable material, e.g., metals, ceramics, semiconductors, organics, polymers, and/or composites. Examples include, but are not limited to, pharmaceutical grade stainless steel, gold, titanium, nickel, iron, gold, tin, chromium, copper, alloys of these or other metals, silicon, silicon dioxide, and polymers, including polymers of hydroxy acids such as lactic acid and glycolic acid polylactide, polyglycolide, polylactide-co-glycolide, and copolymers with polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, or polyesters.

As another example, a skin “patch” may be used to deliver the indicator. Typically, a skin patch includes one or more layers of material that are adhered to the surface of the skin, and can be applied by the subject or another person. Once administered, the indicator may be transported into the skin of the subject, e.g., via diffusion. However, often, the skin patch lacks an external power source. In some cases, the skin patch may also include mechanical elements as well, for example, a cutter such as is discussed below.

As still another example, pressurized fluids may be used to deliver the indicator to the skin, for instance, using a jet injector or a “hypospray.” Typically, such devices produce a high-pressure “jet” of liquid or powder (e.g., a biocompatible liquid, such as saline) that drives material into the skin, and the depth of penetration may be controlled, for instance, by controlling the pressure of the jet. The pressure may come from any suitable source, e.g., a standard gas cylinder or a gas cartridge. A non-limiting example of such a device can be seen in U.S. Pat. No. 4,103,684, issued Aug. 1, 1978, entitled “Hydraulically Powered Hypodermic Injector with Adapters for Reducing and Increasing Fluid Injection Force,” by Ismach. Pressurization of the liquid may be achieved, for example, using compressed air or gas, for instance, from a gas cylinder or a gas cartridge.

In some cases, a cutter able to cut or pierce the surface of the skin may be used to deliver the indicator. For example, the cutter may comprise a hypodermic needle, a knife blade, a piercing element (e.g., a solid or hollow needle), or the like, which can be applied to the skin to create a suitable conduit for the delivery of the indicator into the skin.

The indicator may be delivered to any suitable depth within the skin. For instance, the particles may be delivered to any location within the skin (or below the skin), e.g., to the epidermis, to the dermis, subcutaneously, intramuscularly, etc. In some cases, a “depot” of the indicator may be formed within the skin, and the depot may be temporary or permanent. For instance, the indicator within the depot may eventually degrade (e.g., if the particles are biodegradable), enter the bloodstream, or be sloughed off to the environment. As an example, if the indicator is delivered primarily to the epidermis, the indicator may eventually be sloughed off to the environment (as the epidermis is sloughed off), i.e., such that the indicator is present within the skin of the subject on a temporary basis (e.g., on a time scale of days or weeks). However, if the indicator is delivered to lower layers of tissue, e.g., to the dermis or lower, then the indicator may not be as readily sloughed off to the environment (or the indicator may take longer to be sloughed off into the environment), and thus the indicator may be present in the skin on a longer basis. For instance, the indicator may be present within the subject for weeks, months, or years.

In another set of embodiments, the tracer can be determined externally of the subject, e.g., the indicator may be present externally of the subject. For example, a bodily fluid such as blood or interstitial fluid may be withdrawn from a subject and the tracer determined, e.g. exposed to an indicator present externally of a subject, for example, within a device. Non-limiting examples of suitable devices are disclosed in U.S. patent application Ser. No. 12/716,233, filed Mar. 2, 2010, entitled “Systems and Methods for Creating and Using Suction Blisters or Other Pooled Regions of Fluid within the Skin,” by Levinson, et al.; U.S. patent application Ser. No. 12/716,229, filed Mar. 2, 2010, entitled “Devices and Techniques Associated with Diagnostics, Therapies, and Other Applications, Including Skin-Associated Applications,” by Bernstein, et al.; and U.S. patent application Ser. No. 12/716,226, filed Mar. 2, 2010, entitled “Techniques and Devices Associated with Blood Sampling,” by Levinson, et al., each incorporated herein by reference in its entirety. Examples of such devices are discussed in detail below. Thus, various embodiments of the invention are generally directed to administering a composition comprising a tracer and a substance to a subject, and determining the tracer within the subject, which may be used, in some cases, to determine the substance, for example, its absorption. Other species may be present in the composition as well, e.g., as discussed herein. The substance may be any suitable substance desirably administered to the subject. For instance, the substance may be a drug or other therapeutic agent, or the substance may be a food or a drink desired by the subject, e.g., an alcoholic beverage. In some cases, the substance is an ingestible substance, or can otherwise be taken through the subject's mouth.

Typically, the substance and the tracer are distinct, i.e., the tracer is separate from the substance and is not chemically bonded to the substance, e.g., covalently. However, in certain cases, the substance and the tracer may be chemically bonded together, e.g., by a link that can be degraded (e.g., hydrolyzed) within the body. Such embodiments may be useful, for example, for causing certain pharmacokinetic activities of the tracer to become substantially similar to the pharmacokinetic activities of the substance. The specific link used will depend on both the substance and the tracer. Specific examples of linkers include, but are not limited to, carbon chains (e.g., alkanes or alkenes), polymer units (e.g., synthetic polymers, DNA, RNA, proteins or peptides, etc.), or the like.

The substance may be delivered to the subject using any suitable technique. Thus, for instance, the substance may be administered orally, vaginally, rectally, buccally, pulmonary, topically, nasally, transdermally, through parenteral injection or implantation, via surgical administration, or any other suitable method of administration. The substance may be delivered systemically or locally. The substance may be administered by the subject (i.e., self-administered), or offered or administered to the subject by a different person. Other techniques for administering species to a subject have been discussed above, and can also be used for administering a substance to a subject.

In addition, in some embodiments, one or more of the tracer, the indicator, and/or the substance may be administered simultaneously, although in other embodiments, one or more of these may be administered separately. For example, in one set of embodiments, a carrier containing a tracer and a substance is administered to a subject, and the tracer may be separately administered to the subject or administered along with or in the carrier. The carrier may be, for example, a solid or a liquid, depending on the application. For instance, a solid carrier may be a hard or soft capsule, a pill, a tablet, or a lozenge. A liquid carrier may be, for example, a solution or a suspension in an aqueous or non-aqueous liquid such as a syrup, an elixir, or an emulsion.

In some cases, the carrier may be a pharmaceutically acceptable carrier. In general, pharmaceutically acceptable carriers suitable for use in the invention are well-known to those of ordinary skill in the art. As used herein, a “pharmaceutically acceptable carrier” refers to a non-toxic material that does not significantly interfere with to the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the invention are combined to facilitate the application of the composition. The carrier may be co-mingled or otherwise mixed with one or more of the tracer, indicator, substance, or other species, often in a manner such that there is no interaction which would substantially impair the desired pharmaceutical efficacy or activity. The carrier may be either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some embodiments, tracers and/or indicators may be prepared using pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The formulations may be solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating various compositions without resort to undue experimentation.

Often, one or more of the tracer, the indicator, and/or the substance may be prepared within a kit, before administration to a subject, according to certain embodiments of the invention. A “kit,” as used herein, typically defines a package or an assembly including one or more of the compositions or devices of the invention, and/or other compositions or devices associated with the invention, for example, as previously described. For example, in one set of embodiments, the kit may include a device and one or more compositions for use with the device. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the delivery or use of one or more of the tracer, indicator, substance, other species, or any devices associated with the kit or the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

As mentioned, certain aspects of the present invention are directed to devices able to deliver and/or withdraw fluid from the skin of a subject, as well as methods of use thereof. For example, a bodily fluid such as blood or interstitial fluid may be withdrawn from a subject and exposed to an indicator present externally of a subject, for example, within a device, or a fluid containing an indicator may be administered to the subject. In some cases, the device may pierce the skin of the subject, and fluid can then be delivered and or withdrawn from the subject.

In some cases, more than one fluid transporter system may be present within the device. For instance, the device may be able to be used repeatedly, and/or the device may be able to deliver and/or withdraw fluid at more than one location on a subject, e.g., sequentially and/or simultaneously. In some cases, the device may be able to simultaneously deliver and withdraw fluid to and from a subject.

In some embodiments, the device may take the form of a skin “patch.” Typically, a skin patch includes one or more layers of material that are adhered to the surface of the skin, and can be applied by the subject or another person. In certain embodiments, layers or portions of the skin patch may be removed, leaving other layers or portions behind on the skin. Often, the skin patch lacks an external power source, although the various layers of the patch may contain various chemicals, such as drugs, therapeutic agents, diagnostic agents, reaction entities, etc. In some cases, the skin patch may also include mechanical elements as well, for example, a cutter such as is discussed herein.

In other embodiments, however, the device may be larger. For example, the device may be an electrical and/or a mechanical device applicable or affixable to the surface of the skin, e.g., using adhesive, or other techniques such as those described herein. As another example, the device may be a handheld device that is applied to the surface of the skin of a subject. In some cases, however, the device may be sufficiently small or portable that the subject can self-administer the device. In certain embodiments, the device may also be powered. In some instances, the device may be applied to the surface of the skin, and is not inserted into the skin. In other embodiments, however, at least a portion of the device may be inserted into the skin, for example, mechanically. For example, in one embodiment, the device may include a cutter, such as a hypodermic needle, a knife blade, a piercing element (e.g., a solid or hollow needle), or the like, as discussed herein.

In some cases, the device may be designed such that portions of the device are separable. For example, a first portion of the device may be removed from the surface of the skin, leaving other portions of the device behind on the skin. In one embodiment, a stop may also be included to prevent or control the depth to which the cutter or other device inserts into the skin, e.g., to control penetration to the epidermis, dermis, etc.

Accordingly, as described herein, devices of the invention can be single-stage or multi-stage in some cases. That is, the device can define a single unit that includes one or more components integrally connected to each other which cannot readily be removed from each other by a user, or can include one or more components which are designed to be and can readily be removed from each other. As a non-limiting example of the later, a two-stage patch can be provided for application to the skin of a subject. The patch can include a first stage designed to reside proximate the skin of the subject for the duration of the analysis, which might include an analysis region, a reservoir or other material for creating vacuum or otherwise promoting the flow of fluid or other materials relative to the analysis region, a microneedle to access interstitial fluid via suction blister or without a suction blister or the like. A second stage or portion of the device can be provided that can initiate operation of the device. For example, the two stage device can be applied to the skin of the user. A button or other component or switch associated with the second portion of the device can be activated by the subject to cause insertion of a microneedle to the skin of the subject, or the like. Then, the second stage can be removed, e.g., by the subject, and the first stage can remain on the skin to facilitate analysis. In another arrangement, a two-stage device can be provided where the first stage includes visualization or other signal-producing components and the second stage includes components necessary to facilitate the analysis, e.g., the second stage can include all components necessary to access bodily fluid, transport the fluid (if necessary) to a site of analysis, and the like, and that stage can be removed, leaving only a visualization stage for the subject or another entity to view or otherwise analyze as described herein.

Any or all of the arrangements described herein can be provided proximate a subject, for example on or proximate a subject's skin. Activation of the devices can be carried out as described herein. For example, an on-skin device can be in the form of a patch or the like, optionally including multiple layers for activation, sensing, fluid flow, etc. Activation of the devices can be carried out in a variety of ways. In one manner, a patch can be applied to a subject and a region of the patch activated (e.g., tapped by a user) to inject a microneedle so as to access interstitial fluid. The same or a different tapping or pushing action can activate a vacuum source, open and/or close one or more of a variety of valves, or the like. The device can be a simple one in which it is applied to the skin and operates automatically (where e.g., application to the skin access interstitial fluid and draws interstitial fluid into an analysis region) or the patch or other device can be applied to the skin and one tapping or other activation can cause fluid to flow through administration of a microneedle, opening of a valve, activation of vacuum, or any combination. Any number of activation protocols can be carried out by a user repeatedly pushing or tapping a location or selectively, sequentially, and/or periodically activating a variety of switches (e.g., tapping regions of a patch). With this description, those of ordinary skill in the art can understand how any of the assays described above with respect to one and two can be facilitated. In another arrangement, activation of microneedles, creation of suction blisters, opening and/or closing of valves, and other techniques to facilitate one or more analysis can be carried out electronically or in other manners facilitated by the subject or by an outside controlling entity. For example, a device or patch can be provided proximate a subject's skin and a radio frequency, electromagnetic, or other signal can be provided by a nearby controller or a distant source to activate any of the needles, blister devices, valves or other components of the devices described so that any assay or assays can be carried out as desired.

As discussed, various devices of the invention include various systems and methods for delivering and/or withdrawing fluid from the subject, according to certain embodiments. For instance, the device may comprise a hypodermic needle, a vacuum source, a pressure source, a hygroscopic agent, or the like. Non-limiting examples of suitable delivery techniques include, but are not limited to, injection (e.g., using needles such as hypodermic needles) or a jet injector. For instance, in one embodiment, the fluid is delivered and/or withdrawn manually, e.g., by manipulating a plunger on a syringe. In another embodiment, the fluid can be delivered and/or withdrawn from the skin mechanically or automatically, e.g., using a piston pump or the like. Fluid may also be withdrawn using vacuums such as those discussed herein. For example, vacuum may be applied to a conduit, such as a needle, in fluidic communication with interstitial fluid, e.g., within a pooled region of fluid, in order to draw up at least a portion of the fluid from the pooled region. In yet another embodiment, fluid is withdrawn using capillary action (e.g., using a hypodermic needle having a suitably narrow inner diameter). In still another embodiment, pressure may be applied to force fluid out of the needle. In some embodiments, the device may comprise a mechanism able to cut or pierce the surface of the skin in order to gain access to bodily fluid, or the device may comprise an apparatus for ablating the skin. In some embodiments, fluid may be withdrawn using an electric charge. For example, reverse iontophoresis may be used.

In some embodiments, fluids may be delivered to or withdrawn from the skin using vacuum. The vacuum may be an external vacuum source, and/or the vacuum source may be self-contained within the device. For example, vacuums of at least about 50 mmHg, at least about 100 mmHg, at least about 150 mmHg, at least about 200 mmHg, at least about 250 mmHg, at least about 300 mmHg, at least about 350 mmHg, at least about 400 mmHg, at least about 450 mmHg, at least about 500 mmHg, at least 550 mmHg, at least 600 mmHg, at least 650 mmHg, at least about 700 mmHg, or at least about 750 mmHg may be applied to the skin. As used herein, “vacuum” refers to pressures that are below atmospheric pressure.

As mentioned, any source of vacuum may be used. For example, the device may comprise an internal vacuum source, and/or be connectable to a vacuum source is external to the device, such as a vacuum pump or an external (line) vacuum source. In some cases, vacuum may be created manually, e.g., by manipulating a syringe pump, a plunger, or the like, or the low pressure may be created mechanically or automatically, e.g., using a piston pump, a syringe, a bulb, a Venturi tube, manual (mouth) suction, etc., or the like.

In some cases, the device includes an interface that is able to apply vacuum to the skin. The interface may be, for example, a suction cup or a circular bowl that is placed on the surface of the skin, and vacuum applied to the interface to create a vacuum. In one set of embodiments, the interface is part of a support structure, as discussed herein. The interface may be formed from any suitable material, e.g., glass, rubber, polymers such as silicone, polyurethane, nitrile rubber, EPDM rubber, neoprene, or the like. In some cases, the seal between the interface and the skin may be enhanced (e.g., reducing leakage), for instance, using vacuum grease, petroleum jelly, a gel, or the like. In some cases, the interface may be relatively small, for example, having a diameter of less than about 5 cm, less than about 4 cm, less than about 3 cm, less than about 2 cm, less than about 1 cm, less than about 5 mm, less than about 4 mm, less than about 3 mm, less than about 2 mm, or less than about 1 mm. The interface may be circular, although other shapes are also possible, for example, square, star-shaped (having 5, 6, 7, 8, 9, 10, 11, etc. points), tear-drop, oval, rectangular, or the like. In some cases, non-circular shapes may be used since high-energy points, e.g., the points or corners of the shape may enhance or accelerate blister formation.

The interface may also be selected, in some cases, to keep the size of the pooled region below a certain area, e.g., to minimize pain or discomfort to the subject, for aesthetic reasons, or the like. The interface may be constructed out of any suitable material, e.g., glass, plastic, or the like.

In one set of embodiments, a device of the present invention may not have an external power and/or a vacuum source. In some cases, the device is “pre-loaded” with a suitable vacuum source; for instance, in one embodiment, the device may be applied to the skin and activated in some fashion to create and/or access the vacuum source. As one example, a device of the present invention may be contacted with the skin of a subject, and a vacuum created through a change in shape of a portion of the device (e.g., using a shape memory polymer), or the device may contain one or more sealed, self-contained vacuum compartments, where a seal is punctured in some manner to create a vacuum. For instance, upon puncturing the seal, a vacuum compartment may be in fluidic communication with a needle, which can be used to move the skin towards the device, withdraw fluid from the skin, or the like.

In some cases, the device may be applicable or affixable to the surface of the skin. For example, in one set of embodiments, the device may include a support structure that contains an adhesive that can be used to immobilize the device to the skin. The adhesive may be permanent or temporary, and may be used to affix the device to the surface of the skin. The adhesive may be any suitable adhesive, for example, a pressure sensitive adhesive, a contact adhesive, a permanent adhesive, a cyanoacrylate, glue, gum, hot melts, epoxy, or the like. In some cases, the adhesive is chosen to be biocompatible or hypoallergenic.

In another set of embodiments, the device may be mechanically held to the skin, for example, the device may include mechanical elements such as straps, belts, buckles, strings, ties, elastic bands, or the like. For example, a strap may be worn around the device to hold the device in place against the skin of the subject. In yet another set of embodiments, a combination of these and/or other techniques may be used. As one non-limiting example, the device may be affixed to a subject's arm or leg using adhesive and a strap.

In certain embodiments, the device may also contain an activator. The activator may be constructed and arranged to cause exposure of the fluid transporter to the skin upon activation of the activator. For example, the activator may cause a chemical to be released to contact the skin, a microneedle to be driven into the skin, a vacuum to be applied to the skin, a jet of fluid to be directed to the skin, or the like. The activator may be activated by the subject, and/or by another person (e.g., a health care provider), or the device itself may be self-activating, e.g., upon application to the skin of a subject. The activator may be activated once, or multiple times in some cases.

The device may be activated, for example, by pushing a button, pressing a switch, moving a slider, turning a dial, or the like. The subject, and/or another person, may activate the activator. In some cases, the device may be remotely activated. For example, a health care provider may send an electromagnetic signal which is received by the device in order to activate the device, e.g., a wireless signal, a Bluetooth signal, an Internet signal, a radio signal, etc.

In one set of embodiments, the device may include channels such as microfluidic channels, which may be used to deliver and/or withdraw fluids and/or other materials into or out of the skin, e.g., within the pooled region of fluid. In some cases, the microfluidic channels are in fluid communication with a fluid transporter that is used to deliver and/or withdraw fluids to or from the skin. For example, in one set of embodiments, the device may include a hypodermic needle that can be inserted into skin 10, and fluid may be delivered into the skin via the needle and/or withdrawn from the skin via the needle. The device may also include one or more microfluidic channels to contain fluid for delivery to the needle, e.g., from a source of fluid, and/or to withdraw fluid withdrawn from the skin, e.g., for delivery to an analytical compartment within the device, to a reservoir for later analysis, or the like.

In some cases, more than one compartment may be present within the device, and in some cases, some or all of the compartments may be in fluidic communication, e.g., via channels such as microfluidic channels. In various embodiments, a variety of compartments and/or channels may be present within the device, depending on the application. For example, the device may contain compartments for sensing an analyte, compartments for holding reagents, compartments for controlling temperature, compartments for controlling pH or other conditions, compartments for creating or buffering pressure or vacuum, compartments for controlling or dampening fluid flow, mixing compartments, or the like.

In one set of embodiments, the device may include a sensor, for example embedded within or integrally connected to the device, or positioned remotely but with physical, electrical, and/or optical connection with the device so as to be able to sense a compartment within the device. For example, the sensor may be in fluidic communication with fluid withdrawn from a subject, directly, via a microfluidic channel, an analytical chamber, etc. The sensor may be able to sense a tracer, e.g., one that is suspected of being in a fluid withdrawn from a subject. For example, a sensor may be free of any physical connection with the device, but may be positioned so as to detect the results of interaction of electromagnetic radiation, such as infrared, ultraviolet, or visible light, which has been directed toward a portion of the device, e.g., a compartment within the device. As another example, a sensor may be positioned on or within the device, and may sense activity in a compartment by being connected optically to the compartment. Sensing communication can also be provided where the compartment is in communication with a sensor fluidly, optically or visually, thermally, pneumatically, electronically, or the like, so as to be able to sense a condition of the compartment. As one example, the sensor may be positioned downstream of a compartment, within a channel such a microfluidic channel, or the like.

The sensor may be, for example, a pH sensor, an optical sensor, an oxygen sensor, a sensor able to detect the concentration of a substance, or the like. Non-limiting examples of sensors useful in the invention include dye-based detection systems, affinity-based detection systems, microfabricated gravimetric analyzers, CCD cameras, optical detectors, optical microscopy systems, electrical systems, thermocouples and thermistors, pressure sensors, etc. Those of ordinary skill in the art will be able to identify other sensors for use in the invention. The sensor can include a colorimetric detection system in some cases, which may be external to the device, or microfabricated into the device in certain cases. As an example of a colorimetric detection system, if a dye or a fluorescent entity is used (e.g. in a particle), the colorimetric detection system may be able to detect a change or shift in the frequency and/or intensity of the dye or fluorescent entity.

As described herein, any of a variety of signaling or display methods, associated with analyses, can be provided including signaling visually, by smell, sound, feel, taste, or the like, in one set of embodiments, for example, to indicate the presence of the tracer.

Signal structures include, but are not limited to, displays (visual, LED, light, etc.), speakers, chemical-releasing compartments (e.g., containing a volatile chemical), mechanical devices, heaters, coolers, or the like. In some cases, the signal structure may be integral with the device (e.g., integrally connected with a support structure for application to the skin of the subject, e.g., containing a fluid transporter such as a microneedle).

In one set of embodiments, the device may transmit a signal indicative of the tracer, for example, to be transmitted to another entity for analysis and/or action. For example, a signal can be produced by a device, e.g., based on a sensor reading of a tracer. The signal may represent any suitable data or image. The other entity that the signal is transmitted to can be a human (e.g., a clinician) or a machine. In some cases, the other entity may be able to analyze the signal representing the tracer and take appropriate action. In one arrangement, the other entity is a machine or processor that analyzes the signal and optionally sends a signal back to the device to give direction as to activity (e.g., a cell phone can be used to transmit an image of a signal to a processor which, under one set of conditions, transmits a signal back to the same cell phone giving direction to the user, or takes other action). Other actions can include automatic stimulation of the device or a related device to dispense a medicament or pharmaceutical, or the like. The signal to direct dispensing of a pharmaceutical can take place via the same used to transmit the signal to the entity (e.g., cell phone) or a different vehicle or pathway. Telephone transmission lines, wireless networks, Internet communication, and the like can also facilitate communication of this type.

According to various sets of embodiments, the device may be used one, or multiple times, depending on the application. For instance, obtaining samples for sensing, according to certain embodiments of the invention, can be done such that sensing can be carried out continuously, discretely, or a combination of these. For example, where a bodily fluid such as interstitial fluid is accessed for determination of an analyte, fluid can be accessed discretely (i.e., as a single dose, once or multiple times), or continuously by creating a continuous flow of fluid which can be analyzed once or any number of times. Additionally, testing can be carried out once, at a single point in time, or at multiple points in time, and/or from multiple samples (e.g., at multiple locations relative to the subject).

Alternatively or in addition, testing can be carried out continuously over any number of points in time involving one or any number of locations relative to the subject or other multiple samples. As an example, one bolus or isolated sample, of fluid such as interstitial fluid can be obtained. From that fluid a test can be carried out to determine whether a particular analyte or other agent exists in the fluid. Alternatively, two or more tests can be carried out involving that quantity of fluid to determine the presence and/or quantity of two or more analytes, and any number of such tests can be carried out. Tests involving that quantity of fluid can be carried out simultaneously or over a period of time. For example, a test for a particular analyte can be carried out at various points in time to determine whether the result changes over time, or different analytes can be determined at different points in time. As another example, a pool of fluid can be formed between layers of skin via, e.g., a suction blister and either within the suction blister or from fluid drawn from the suction blister and placed elsewhere, any of the above and other analysis can be carried out at one or more points in time. Where a suction blister is formed in such a way that interstitial fluid within the blister changes over time (where an equilibrium exists between interstitial fluid within the subject and interstitial fluid in the suction blister itself, i.e., the fluid within the blister is ever changing to reflect the content of the interstitial fluid of the subject in the region of the blister over time). Testing of fluid within or from the suction blister at various points in time can provide useful information.

As mentioned, certain aspects of the present invention are generally directed to particles such as anisotropic particles or colloids, which can be used as indicators in some embodiments of the invention. The particles may include microparticles and/or nanoparticles. As discussed above, a “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer. The particles may be spherical or non-spherical, in some cases. For example, the particles may be oblong or elongated, or have other shapes such as those disclosed in U.S. patent application Ser. No. 11/851,974, filed Sep. 7, 2007, entitled “Engineering Shape of Polymeric Micro- and Nanoparticles,” by S. Mitragotri, et al.; International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008; U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007, each of which is incorporated herein by reference.

An “anisotropic” particle, as used herein, is one that is not spherically symmetric (although the particle may still exhibit various symmetries), although the particle may have sufficient asymmetry to carry out at least some of the goals of the invention as described herein. On the basis of the present disclosure, this will be clearly understood by those of ordinary skill in the art. The asymmetry can be asymmetry of shape, of composition, or both. As an example, a particle having the shape of an egg or an American football is not perfectly spherical, and thus exhibits anisotropy. As another example, a sphere painted such that exactly one half is red and one half is blue (or otherwise presents different surface characteristics on different sides) is also anisotropic, as it is not perfectly spherically symmetric, although it would still exhibit at least one axis of symmetry.

Accordingly, a particle may be anisotropic due to its shape and/or due to two or more regions that are present on the surface of and/or within the particle. For instance, the particle may include a first surface region and a second surface region that is distinct from the first region in some way, e.g., due to coloration, surface coating, the presence of one or more reaction entities, etc. The particle may include different regions only on its surface or the particle may internally include two or more different regions, portions of which extend to the surface of the particle. The regions may have the same or different shapes, and be distributed in any pattern on the surface of the particle. For instance, the regions may divide the particle into two hemispheres, such that each hemisphere has the same shape and/or the same surface area, or the regions may be distributed in more complex arrangements.

Non-limiting examples of particles can be seen in U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007; or U.S. Provisional Patent Application Ser. No. 61/058,796, filed Jun. 4, 2008, entitled “Compositions and Methods for Diagnostics, Therapies, and Other Applications,” by D. Levinson, each of which is incorporated herein by reference.

The particles (which may be anisotropic, or not anisotropic) may be formed of any suitable material, depending on the application. For example, the particles may comprise a glass, and/or a polymer such as polyethylene, polystyrene, silicone, polyfluoroethylene, polyacrylic acid, a polyimide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the particles may comprise a ceramic such as tricalcium phosphate, hydroxyapatite, fluorapatite, aluminum oxide, or zirconium oxide. In some cases (for example, in certain biological applications), the particles may be formed from biocompatible and/or biodegradable polymers such as polylactic and/or polyglycolic acids, polyanhydride, polycaprolactone, polyethylene oxide, polyacrylamide, polyacrylic acid, polybutylene terephthalate, starch, cellulose, chitosan, and/or combinations of these. In one set of embodiments, the particles may comprise a hydrogel, such as agarose, collagen, or fibrin. The particles may include a magnetically susceptible material in some cases, e.g., a material displaying paramagnetism or ferromagnetism. For instance, the particles may include iron, iron oxide, magnetite, hematite, or some other compound containing iron, or the like. In another embodiment, the particles can include a conductive material (e.g., a metal such as titanium, copper, platinum, silver, gold, tantalum, palladium, rhodium, etc.), or a semiconductive material (e.g., silicon, germanium, CdSe, CdS, etc.). Other particles potentially useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, or GaAs. The particles may include other species as well, such as cells, biochemical species such as nucleic acids (e.g., RNA, DNA, PNA, etc.), proteins, peptides, enzymes, nanoparticles, quantum dots, fragrances, indicators, dyes, fluorescent species, chemicals, small molecules (e.g., having a molecular weight of less than about 1 kDa), or the like.

As an example, certain particles or colloids such as gold nanoparticles can be coated with agents capable of interacting with a tracer. Such particles may associate with each other, or conversely, dissociate in the presence of a tracer in such a manner that a change is conferred upon the light absorption property of the material containing the particles. This approach can also be used as a skin-based visual sensor, in one embodiment. A non-limiting example of a technique for identifying aggregates is disclosed in U.S. patent application Ser. No. 09/344,667, filed Jun. 25, 1999, entitled “Nanoparticles Having Oligonucleotides Attached Thereto and Uses Therefor,” by Mirkin, et al., now U.S. Pat. No. 6,361,944, issued Mar. 26, 2002.

The particles may also have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. As discussed, the particles may be spherical or non-spherical. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous. Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks, and the like. See, e.g., International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008, incorporated herein by reference.

In one aspect of the invention, a particle may include one or more reaction entities present on the surface (or at least a portion of the surface) of the particle. The reaction entity may be any entity able to interact with and/or associate with a tracer, or another reaction entity. For instance, the reaction entity may be a binding partner able to bind an tracer. For example, the reaction entity may be a molecule that can undergo binding with a particular tracer. The reaction entities may be used, for example, to determine pH or metal ions, proteins, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other compounds of interest.

Thus, the invention provides, in certain embodiments, particles that are able to bind to a tracer, e.g., via a binding partner to the tracer, and such particles can be used to determine the tracer. In one set of embodiments, more than one particle may be able to bind a tracer, and/or more than one tracer may bind to a particle. In addition, more than one tracer may be determined in a subject, e.g., through the use of different particle types and/or through the use of particles able to determine more than one tracer, such as those discussed above. For instance, a first set of particles may determine a first tracer and a second set of particles may determine a second tracer.

In some cases, such multiple binding properties may result in the clustering of more than one particle to a tracer and/or more than one tracer to a particle. Such clustering can be determined in some fashion, e.g., via a change in an optical property.

As an example, an aggregate of particles may form in the absence of a tracer, but disaggregate (at least partially) in the presence of the tracer, e.g., if the tracer and the particles exhibit competitive or non-competitive inhibition. Such binding and/or aggregation may be equilibrium-based in some cases, i.e., the binding and/or aggregation occurs in equilibrium with unbinding or disaggregation processes. Thus, when the environment surrounding the particles is altered in some fashion (e.g., a change in concentration of an tracer), the equilibrium may shift in response, which can be readily determined (e.g., as a change in color). It should be noted that such equilibrium-based systems may be able to determine such changes in environment, in some cases, without the need to apply any energy to determine the environmental change. In another example, aggregation may cause a change in an electrical or a magnetic property.

As an example, an optical property of the medium containing the clusters may be altered in some fashion (e.g., exhibiting different light scattering properties, different opacities, different degrees of transparency, etc.), which can be correlated with the tracer. In some cases, the color may change in intensity, for example, the clustering of particles may bring two or more reactants into close proximity.

Other properties may also be determined besides color. Accordingly, it should be understood that the use of “color” with respect to particles as used herein is by way of example only, and other properties may be determined instead of or in addition to color. For instance, clustering of aniostropic particles may cause a change in an electrical or a to magnetic property of the particles, which can be determined by determining an electrical or a magnetic field. As another example, the first region and the second region may have different reactivities (e.g., the first region may be reactive to an enyzme, an antibody, etc.), and aggregation of the particles may cause a net change in the reactivity. As still another example, size may be used to determine the particles and/or the tracer. For instance, the aggregates may be visually identifiable, the aggregates may form a precipitant, or the like. Thus, for example, the particles (which may be anisotropic or not anisotropic) may appear to be a first color when separate, and a second color when aggregation occurs. In some cases, an assay (e.g., an agglutination assay) may be used to determine the aggregation. In another set of embodiments, an ordering of the particles may be determined. For example, in the absence of a tracer, the particles may be ordered on the surface of a substrate; while in the presence of a tracer, the particles may bind to the tracer and become disordered relative to the surface. This ordering may be determined, for example, as a change in an optical property of the surface (e.g., index of refraction, color, opacity, etc.). As yet other examples, a shape change may be produced using a shape memory polymer or a “smart polymer,” and this may be able to be sensed by feel. Alternatively, a color may be released, a hydrolysis reaction may occur, or aggregation of the particles may occur.

In one embodiment, the binding or presence of a tracer results in a tactile change (e.g., change in shape or texture). For example, shape memory polymer (SMPs) can be used to detect the presence of one or more tracers. SMPs are generally characterized as phase segregated linear block co-polymers having a hard segment and a soft segment. The hard segment is typically crystalline, with a defined melting point, and the soft segment is typically amorphous, with a defined glass transition temperature. In some embodiments, however, the hard segment is amorphous and has a glass transition temperature rather than a melting point. In other embodiments, the soft segment is crystalline and has a melting point rather than a glass transition temperature. The melting point or glass transition temperature of the soft segment is substantially less than the melting point or glass transition temperature of the hard segment.

When the SMP is heated above the melting point or glass transition temperature of the hard segment, the material can be shaped. This (original) shape can be “memorized” by cooling the SMP below the melting point or glass transition temperature of the hard segment. When the shaped SMP is cooled below the melting point or glass transition temperature of the soft segment while the shape is deformed, that (temporary) shape is fixed. The original shape is recovered by heating the material above the melting point or glass transition temperature of the soft segment but below the melting point or glass transition temperature of the hard segment. The recovery of the original shape, which is induced by an increase in temperature, is called the thermal shape memory effect. Properties that describe the shape memory capabilities of a material include the shape recovery of the original shape and the shape fixity of the temporary shape

Shape memory polymers can contain at least one physical crosslink (physical interaction of the hard segment) or contain covalent crosslinks instead of a hard segment. The shape memory polymers also can be interpenetrating networks or semi-interpenetrating networks. In addition to changes in state from a solid to liquid state (melting point or glass transition temperature), hard and soft segments may undergo solid to solid state transitions, and can undergo ionic interactions involving polyelectrolyte segments or supramolecular effects based on highly organized hydrogen bonds.

Other polymers that can change shape or phase as a function of temperature include PLURONICS®. These are also known as poloxamers, nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist that have slightly different properties. For the generic term “poloxamer,” these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the PLURONICS® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content).

Other temperature sensitive polymers that form gels that have a distinct phase change at its lower critical solution temperature (LCST) including the cross-linked copolymers comprising hydrophobic monomers, hydrogen bonding monomers, and thermosensitive monomers.

Additional thermal responsive, water soluble polymers including the co-polymerization product of N-isopropyl acrylamide (NIP); 1-vinyl-2-pyrrolidinone (VPD); and optionally, acrylic acid (AA), change shape as a function of temperature. As the proportion of component AA increases, the Lower Critical Solution Temperature (LCST) decreases and the COOH reactive groups increase, which impart high reactivity to the copolymer. By adjusting the proportion of the monomers, a broad range of LCST can be manipulated from about 20° C. to 80° C.

While the shape memory effect is typically described in the context of a thermal effect, the polymers can change their shape in response to application of light, changes in ionic concentration and/or pH, electric field, magnetic field or ultrasound. For example, a SMP can include at least one hard segment and at least one soft segment, wherein at least two of the segments, e.g., two soft segments, are linked to each other via a functional group that may be cleavable under application of light, electric field, magnetic field or ultrasound. The temporary shape may be fixed by crosslinking the linear polymers. By cleaving those links the original shape can be recovered. The stimuli for crosslinking and cleaving these bonds can be the same or different.

In one embodiment, the shape memory polymer composition binds, complexes to, or interacts with a tracer, which can be a chromophore. The hard and/or soft segments can include double bonds that shift from cis to trans isomers when the chromophores absorb light. Light can therefore be used to detect the presence of a chromophore tracer by observing whether or not the double bond isomerizes.

The shape memory effect can also be induced by changes in ionic strength or pH. Various functional groups are known to crosslink in the presence of certain ions or in response to changes in pH. For example, calcium ions are known to crosslink amine and alcohol groups, i.e., the amine groups on alginate can be crosslinked with calcium ions. Also, carboxylate and amine groups become charged species at certain pHs. When these species are charged, they can crosslink with ions of the opposite charge. The presence of groups, which respond to changes in the concentration of an ionic species and/or to changes in pH, on the hard and/or soft segments results in reversible linkages between these segments. One can fix the shape of an object while crosslinking the segments. After the shape has been deformed, alteration of the ionic concentration or pH can result in cleavage of the ionic interactions which formed the crosslinks between the segments, thereby relieving the strain caused by the deformation and thus returning the object to its original shape. Because ionic bonds are made and broken in this process, it can only be performed once. The bonds, however, can be re-formed by altering the ionic concentration and/or pH, so the process can be repeated as desired. Thus, in this embodiment, the presence of a tracer which changes the ionic strength or pH can induce a shape memory effect in the polymer confirming the presence of the tracer.

Electric and/or magnetic fields can also be used to induce a shape memory effect. Various moieties, such as chromophores with a large number of delocalized electrons, increase in temperature in response to pulses of applied electric or magnetic fields as a result of the increased electron flow caused by the fields. After the materials increase in temperature, they can undergo temperature induced shape memory in the same manner as if the materials were heated directly. These compositions are useful in biomedical applications where the direct application of heat to an implanted material may be difficult, but the application of an applied magnetic or electric field would only affect those molecules with the chromophore, and not heat the surrounding tissue. For example, the presence of a chromophore tracer with a large number of delocalized electrons can be cause an increase in temperature in the microenvironment surrounding the shape memory polymer implant in response to pulses of applied electric or magnetic fields. This increase in temperature can in turn cause a thermal shape memory effect, thus confirming the presence of a particular tracer.

Other types of “smart polymers” may also be used. The combination of the capabilities of stimuli-responsive components such as polymers and interactive molecules to form site-specific conjugates are useful in a variety of assays, separations, processing, and other uses. The polymer chain conformation and volume can be manipulated through alteration in pH, temperature, light, or other stimuli. The interactive molecules can be biomolecules like proteins or peptides, such as antibodies, receptors, or enzymes, polysaccharides or glycoproteins which specifically bind to ligands, or nucleic acids such as antisense, ribozymes, and aptamers, or ligands for organic or inorganic molecules in the environment or manufacturing processes. The stimuli-responsive polymers are coupled to recognition biomolecules at a specific site so that the polymer can be manipulated by stimulation to alter ligand-biomolecule binding at an adjacent binding site, for example, the biotin binding site of streptavidin, the antigen-binding site of an antibody or the active, substrate-binding site of an enzyme. Binding may be completely blocked (i.e., the conjugate acts as an on-off switch) or partially blocked (i.e., the conjugate acts as a rheostat to partially block binding or to block binding only of larger ligands). Once a ligand is bound, it may also be ejected from the binding site by stimulating one (or more) conjugated polymers to cause ejection of the ligand and whatever is attached to it. Alternatively, selective partitioning, phase separation or precipitation of the polymer-conjugated biomolecule can be achieved through exposure of the stimulus-responsive component to an appropriate environmental stimulus.

Liquid crystal polymeric materials can also be used to provide a signal for detection or quantification of tracer. Liquid crystals are materials that exhibit long-range order in only one or two dimensions, not all three. A distinguishing characteristic of the liquid crystalline state is the tendency of the molecules, or mesogens, to point along a common axis, known as the director. This feature is in contrast to materials where the molecules are in the liquid or amorphous phase, which have no intrinsic order, and molecules in the solid state, which are highly ordered and have little translational freedom. The characteristic orientational order of the liquid crystal state falls between the crystalline and liquid phases. These can be pressure or temperature sensitive, and react by producing a change in color or shape.

In one set of embodiments, at least some of the particles used in the subject to determine the tracer are anisotropic particles (in other cases, however, the particles are not necessarily anisotropic), and in some cases, substantially all of the particles are anisotropic particles. In certain cases, at least about 10%, at least about 30%, at least about 40%, at least about 50%, at least about 60%, at least about 70%, at least about 80%, at least about 90%, at least about 95%, or at least about 99% of the particles are anisotropic particles. In one embodiment, the anisotropic particles may have a first region having a first color and a second region having a second color distinct from the first color, and the particles, upon exposure to the tracer within the subject, may form clusters that exhibit an excess of the second region or second color relative to the first region or first color, as discussed above. The particles may be present, for example, in the bloodstream and/or within the skin of the subject.

In some cases, the particles after delivery into the skin may give the appearance of a “tattoo” or a permanent mark within the skin, and the tattoo or other mark may be of any color and/or size. For instance, in one embodiment, anisotropic particles such as those described above that are able to bind glucose may be delivered into the skin of a subject, and such particles, after deposition within the skin may react to the presence or absence of a tracer by exhibiting a change in color. The particles may exhibit a color change based on the presence or absence of tracer, and/or the concentration of tracer. For instance, the particles may exhibit a first color (e.g., green) when not aggregated, and a second color (e.g., red or brown) when aggregated, or the particles may be invisible when not aggregated, but visible (e.g., exhibiting a color) when aggregated. The particles may be, for example, anisotropic particles having a first surface region having a first color (e.g., green) and a second surface region having a second color (e.g., red), and the first surface region may contain a binding partner to glucose. At low levels of tracer, the particles may exhibit a combination of the first and second colors, while at higher levels of tracer, the particles may exhibit more of the second color.

In some cases, the particles may be suspended in a carrying fluid, e.g., saline, or the particles may be contained within a matrix, e.g., a porous matrix that is or becomes accessible by interstitial fluid after delivery, or a hydrogel matrix, etc. For instance, the matrix may be formed from a biodegradable and/or biocompatible material such as polylactic acid, polyglycolic acid, poly(lactic-co-glycolic acid), etc., or other similar materials.

In some cases, the matrix may prevent or at least inhibit an immunological response by the subject to the presence of the particles, while allowing equilibration of tracers, analytes, etc. with the particles to occur, e.g., if the matrix is porous. For instance, the pores of a porous matrix may be such that immune cells are unable to penetrate, while proteins, small molecules (e.g., glucose, ions, dissolved gases, etc.) can penetrate. The pores may be, for instance, less than about 5 micrometers, less than about 4 micrometers, less than about 3 micrometers, less than about 2 micrometers, less than about 1.5 micrometers, less than about 1.0 micrometers, less than about 0.75 micrometers, less than about 0.6 micrometers, less than about 0.5 micrometers, less than about 0.4 micrometers, less than about 0.3 micrometers, less than about 0.1 micrometers, less than about 0.07 micrometers, and in other embodiments, or less than about 0.05 micrometers. The matrix may comprise, for example, biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these, and/or other materials such as agarose, collagen, fibrin, or the like.

Thus, in one set of embodiments, particles are provided which can be analogized to a light on an automotive dashboard, e.g., green for normal, yellow for suspicious, slightly low or slightly high, and red for abnormal. The subject then knows that they need to be seen, and the degree of urgency, by appropriate medical personnel. The particles may be placed and read at the site of detection. For example, the devices may provide a visual colorimetic signal, but other signals are possible, such as smell (released upon change in pH or temperature, for example), or tactile (shape change due to chemical reaction).

Signals from the particles can be used to generate a pattern or color which is indicative of the presence and/or amount of tracer. The density, shape, color, or intensity of the pattern or color may provide a yes-no type answer or may be graduated to provide quantitative amounts. This could also be effected by exposure to a pH or temperature change in some embodiments. Other patterns include, for example, + and − signs, arrows (e.g., up arrows or down arrows), faces (smiley, neutral, sad), etc., or the like.

In one set of embodiments, the skin surface may change in feel when there is a reaction. For example, shape memory polymers may say “OK” when the cholesterol level is below 150 mg/dl. These may change to ready “HIGH” when the cholesterol level exceeds 200 mg/dl. The device may be blank or lack definition at values between these levels.

The particles may change when reacted with a tracer. This may result in a smell such as a food odor being released as a function of a pH or temperature change. For example, an encapsulated scent may be released. In some cases, FDA GRAS ingredients may be used as signals.

These may be applied to the skin to measure a change in temperature indicative of disease or inflammation. In one embodiment, the device may be colorless or a color indicative of normal temperature (for example, green), or the device will display a message such as “OK.” In the event the temperature exceeds a certain level, such as 101° F., the color changes (for example, yellow for caution or red for warning or critical) or the message changes (for example, if shape memory polymers are used) to read “HOT.” These are particularly useful in a setting such as a day care, where there are a number of babies or young children to supervise, and fevers can occur rapidly.

In another embodiment, the particles may be used to measure a decrease in blood oxygen, or measure the amount of molecules such as glucose, cholesterol, triglycerides, cancer markers, or infectious agents, by providing agents that specifically react with the molecules, and signal generating agents which produces signal in an amount correlated with the amounts of the molecules that react. As another non-limiting example, analogous to the temperature monitor, a pre-set level can be used to create a message that says “C high,” for example, or “insulin!” for example, which effects a color change.

As discussed above, the devices may, instead of a color change or message change, change shape, emit a scent or flavor, or otherwise notify the person of a need to seek further information. In some cases, this might be to seek medical attention where the indicator of a disorder can be confirmed and appropriate medical intervention obtained. In the case of temperature indicative of a fever, the caregiver might measure the temperature using a standard thermometer. In the case of a hormone change, indicative of pregnancy or ovulation, an ELISA test might be performed using a urine sample. In the case of high glucose, this could be confirmed using a standard glucose monitor and a blood sample.

In one aspect of the present invention, methods of forming particles such as those described herein are provided. For instance, in one set of embodiments, electrospraying or electrospinning techniques are used to prepare particles. In some cases, two or more fluid streams (including liquid jets) are combined together such that the two or more fluid streams contact over spatial dimensions sufficient to form a composite stream. In some cases, there is little or no mixing of the two or more fluid streams within the composite stream. In some variations, the fluid streams are electrically conductive, and in certain cases, a cone-jet may be formed by combining the two or more fluid streams under the influence of an electric field.

In some cases, the composite stream is directed at a substrate, e.g., by the application of a force field such as an electric field. For instance, if the composite stream is charged, an electric field may be used to urge the composite stream towards a substrate. The composite stream may be continuous or discontinuous in some cases, e.g., forming a series of droplets (which may be spherical or non-spherical). In some cases, the composite stream is hardened prior to and/or upon contact with the substrate. For example, the composite stream may be urged towards the substrate under conditions in which at least a portion of the composite stream (e.g., a solvent) is able to evaporate, causing the remaining stream to harden and/or precipitate, e.g., to form particles, spheres, rods, fibers, or the like. In some variations, the composite stream fragments in droplets that can lead to particle, sphere, rod, and/or fiber formation.

Additional examples of techniques for forming such particles or fibers can be found in U.S. patent application Ser. No. 11/272,194, filed Nov. 10, 2005, entitled “Multi-Phasic Nanoparticles,” by Lahann, et al., published as U.S. Patent Application Publication No. 2006/0201390 on Sep. 14, 2006; or priority to U.S. patent application Ser. No. 11/763,842, filed Jun. 15, 2007, entitled “Multiphasic Biofunctional Nano-Components and Methods for Use Thereof,” by Lahann, published as U.S. Patent Application Publication No. 2007/0237800 on Oct. 11, 2007, each of which is incorporated herein by reference.

In one set of embodiments, solvent evaporation techniques may be used. In one embodiment, a polymer may be dissolved in a volatile organic solvent, such as methylene chloride. Drugs or other suitable species are added to the solution, and the mixture is suspended in an aqueous solution that contains a surface active agent such as poly(vinyl alcohol). The resulting emulsion can be stirred until most of the organic solvent evaporated, leaving solid particles. The resulting particles may be washed with water and dried overnight in a lyophilizer. Particles with different sizes or morphologies can be obtained by this method. This method is useful for relatively stable polymers like polyesters and polystyrene.

In another set of embodiments, solvent removal techniques may be used, e.g., for polymers such as polyanhydrides. In one embodiment, a polymer may be dissolved in a volatile organic solvent like methylene chloride. The mixture can be suspended by stirring in an organic oil (such as silicon oil) to form an emulsion. This can be used to make particles from polymers with high melting points and different molecular weights. Particles that range, for example, between 1-2000 microns, 1-1000 microns, 1-500 microns, 1-300 microns, 1-100 microns, 1-30 microns, 1-10 microns, etc. in diameter can be obtained by this procedure. The external morphology of spheres produced with this technique may be controlled by controlling the type of polymer used.

In yet another set of embodiments, spray-drying techniques may be used. In one embodiment, a polymer is dissolved in organic solvent. The solution or the dispersion is then spray-dried. Particles ranging between, for example, 1-2000 microns, 1-1000 microns, 1-500 microns, 1-300 microns, 1-100 microns, 1-30 microns, 1-10 microns, etc. in diameter can be obtained with a morphology which depends on the type of polymer used.

In still another set of embodiments, interfacial polycondensation techniques may be used. In one embodiment, a monomer is dissolved in a solvent. A second monomer is dissolved in a second solvent (typically aqueous) which is immiscible with the first. An emulsion may be formed by suspending the first solution through stirring in the second solution. Once the emulsion is stabilized, an initiator can be added to the aqueous phase causing interfacial polymerization at the interface of each droplet of emulsion.

In yet another set of embodiments, phase inversion techniques may be used. In one set of embodiments, particles can be formed from polymers using a phase inversion method wherein a polymer is dissolved in a solvent and the mixture is poured into a non-solvent for the polymer, to spontaneously produce particles under favorable conditions. The method can be used to produce particles in a wide range of diameters, including, for example, about 100 nanometers to about 10 microns. Examples of polymers which can be used include polyvinylphenol and polylactic acid. In some cases, the polymer can be dissolved in an organic solvent and then contacted with a non-solvent, which causes phase inversion of the dissolved polymer to form particles, optionally incorporating an antigen or other substance.

In still another set of embodiments, phase separation techniques may be used. In one set of embodiments, the polymer is dissolved in a solvent to form a polymer solution. While continually stirring, a nonsolvent for the polymer may be added to the solution to decrease the polymer's solubility. Depending on the solubility of the polymer in the solvent and nonsolvent, the polymer may precipitate and/or phase separate into a polymer-rich and a polymer-poor phase. Under proper conditions, the polymer in the polymer-rich phase may migrate to the interface with the continuous phase, forming particles.

In yet another set of embodiments, spontaneous emulsification techniques can be used. One set of embodiments involves solidifying emulsified liquid polymer droplets by changing temperature, evaporating solvent, and/or adding chemical cross-linking agents. In still another set of embodiments, hot melt techniques may be used.

In some cases, the particles may comprise a gel. For instance, in one set of embodiments, particles made of gel-type polymers, such as alginate and hyaluronic acid, can be produced through ionic gelation techniques. In one embodiment, polymers can be first dissolved in an aqueous solution and then extruded through a droplet forming device, which in some instances employs a flow of nitrogen and/or other gases to break off the droplet. A slowly stirred (approximately 100-170 RPM) ionic hardening bath may be positioned below the extruding device to catch the forming droplets. The particles are left to incubate in the bath to allow gelation to occur. Particle size may be controlled, for example, by using various size extruders or varying nitrogen gas or polymer solution flow rates. In one embodiment, chitosan particles can be prepared by dissolving the polymer in acidic solution and crosslinking it with tripolyphosphate. In another embodiment, carboxymethyl cellulose (CMC) nanoparticles can be prepared by dissolving the polymer in acid solution and precipitating the nanoparticle with lead ions. In some cases where negatively charged polymers (e.g., alginate, CMC) are used, positively charged ligands (e.g., polylysine, polyethyleneimine) of different molecular weights can be ionically attached.

Other methods known in the art that can be used to prepare nanoparticles include, but are not limited to, polyelectrolyte condensation, single and double emulsion (probe sonication), nanoparticle molding, or electrostatic self-assembly (e.g., polyethylene imine-DNA or liposomes).

In some cases, the particles may include functional groups used to bind or complex the tracer, and such functional groups can be introduced prior to particle formation (e.g., monomers can be functionalized with one or more functional groups for binding or complexing the tracer) or the functional groups can be introduced after particle formation (e.g., by functionalizing the surface of the microparticle with reactive functional groups). The particles may optionally have encapsulated therein one or more core materials. In one embodiment, the particles may be present in an effective amount to provide a signal detectable to the user without the need for additional equipment. For example, the articles should be present in an effective amount to provide a change in taste, smell, shape, and/or color upon binding or complexing the tracer that is easily detectable by the user.

U.S. Provisional Patent Application Ser. No. 61/058,796, filed Jun. 4, 2008, entitled “Compositions and Methods for Diagnostics, Therapies, and Other Applications,” by D. Levinson, is incorporated herein by reference. Also incorporated herein by reference are U.S. Provisional Patent Application Ser. No. 61/163,710, filed on Mar. 26, 2009, entitled “Systems and Methods for Creating and Using Suction Blisters or Other Pooled Regions of Fluid Within the Skin,” by D. Levinson; U.S. Provisional Patent Application Ser. No. 61/163,750, filed on Mar. 26, 2009, entitled “Monitoring of Implants and Other Devices,” by D. Levinson, et al.; U.S. Provisional Patent Application Ser. No. 61/163,793, filed on Mar. 26, 2009, entitled “Compositions and Methods for Diagnostics, Therapies, and other Applications,” by D. Levinson; U.S. Provisional Patent Application Ser. No. 61/163,791, filed on Mar. 26, 2009, entitled “Compositions and Methods for Rapid One-Step Diagnosis,” by D. Levinson; U.S. Provisional Patent Application Ser. No. 61/163,733, filed Mar. 26, 2009, entitled “Determination of Tracers within Subjects,” by Levinson; U.S. Provisional Patent Application Ser. No. 61/269,436, filed Jun. 24, 2009, entitled “Devices and Techniques Associated with Diagnostics, Therapies, Other Applications, Including Skin-Associated Applications,” by Levinson, et al.; U.S. patent application Ser. No. 12/716,233, filed Mar. 2, 2010, entitled “Systems and Methods for Creating and Using Suction Blisters or Other Pooled Regions of Fluid within the Skin,” by Levinson, et al.; U.S. patent application Ser. No. 12/716,229, filed Mar. 2, 2010, entitled “Devices and Techniques Associated with Diagnostics, Therapies, and Other Applications, Including Skin-Associated Applications,” by Bernstein, et al.; and U.S. patent application Ser. No. 12/716,226, filed Mar. 2, 2010, entitled “Techniques and Devices Associated with Blood Sampling,” by Levinson, et al.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
providing a substance, the presence and/or amount of which is to be determined in association with a subject;
offering, to the subject, a composition comprising the substance and a tracer able to be introduced into and be determined in association with the subject; and
determining whether the substance was administered to the subject by determining the tracer in association with the subject.

2. The method of claim 1, wherein the substance and the tracer are present in a composition offered to the subject.

3. The method of claim 1, wherein the substance is a drug.

4-5. (canceled)

6. The method of claim 1, wherein the composition is a liquid containing the tracer and the substance.

7. The method of claim 1, wherein the composition is a solid.

8-9. (canceled)

10. The method of claim 1, wherein the tracer and the substance are chemically unbonded to each other.

11. The method of claim 1, wherein the tracer and the substance are chemically bonded to each other.

12. (canceled)

13. The method of claim 1, comprising wherein the pharmacokinetic absorption profile of the tracer and the substance are substantially similar.

14. The method of claim 1, wherein the composition is administered orally to the subject.

15. The method of claim 1, wherein the composition is administered parenterally to the subject.

16-17. (canceled)

18. The method of claim 1, wherein the indicator comprises particles.

19-38. (canceled)

39. The method of claim 1, wherein the tracer comprises a protein.

40. The method of claim 1, wherein the tracer comprises inulin.

41. The method of claim 1, wherein the tracer has a molecular weight of less than about 1000 Da.

42. The method of claim 1, wherein the tracer comprises creatinine.

43-44. (canceled)

45. The method of claim 1, wherein determining the tracer in association with the subject comprises quantitatively determining the tracer.

46. A method, comprising:
administering a composition comprising a tracer and a substance to a subject; and
determining activity of the substance within the subject by determining the tracer within the subject.

47. The method of claim 46, wherein the substance is a drug.

48. (canceled)

49. The method of claim 46, wherein the tracer and the substance are chemically unbonded to each other.

50-52. (canceled)

53. The method of claim 46, wherein the composition is administered orally to the subject.

54-56. (canceled)

57. The method of claim 55, wherein the indicator comprises particles.

58-61. (canceled)

62. The method of claim 46, comprising determining the pharmokinetic absorption profile of the drug within the subject.

63. A method, comprising;
administering a composition comprising a tracer and a pharmaceutically acceptable carrier to a subject; and
administering an indicator responsive to the tracer to the skin of the subject.

64. (canceled)

65. The method of claim 63, wherein the indicator comprises particles.

66-68. (canceled)

69. The method of claim 63, wherein the composition is administered orally to the subject.

70. The method of claim 63, wherein the composition is administered parenterally to the subject.

71. The method of claim 63, wherein the drug and the tracer exhibit substantially the same pharmacokinetic absorption.

72. The method of claim 63, further comprising determining the indicator after administering the indicator.

73. (canceled)

74. A method, comprising:
administering, to a subject, a composition comprising a tracer and a substance able to be introduced and be determined in the subject; and
administering an indicator responsive to the tracer to the skin of the subject, the indicator able to change a property upon response to the tracer.

75. The method of claim 74, wherein the property is a visual property.

76. (canceled)

77. The method of claim 74, wherein the indicator comprises particles.

78. The method of claim 77, wherein the property is aggregation of particles.

79. (canceled)

80. The method of claim 74, wherein the composition further comprises a drug.

81-87. (canceled)

88. A method, comprising:
offering, to a subject, an ingestible composition comprising a tracer and an ingestible substance, and a dermally-applicable composition comprising an indicator able to change a visual property upon exposure to the tracer.

89. (canceled)

90. The method of claim 88, wherein the substance is a drug.

91-97. (canceled)

98. The method of claim 88, wherein the indicator comprises particles.

99. (canceled)

100. A method, comprising:
determining a substance within a subject by monitoring a tracer within the subject.

101. The method of claim 100, wherein the tracer and the substance are chemically unbonded to each other.

102-103. (canceled)

104. The method of claim 100, further comprising simultaneously administering the tracer and the active ingredient to the subject.

105. The method of claim 100, wherein the substance is a drug.

106. (canceled)

107. The method of claim 100, wherein an indicator is used to monitor the tracer.

108-111. (canceled)

112. A method, comprising:
determining an exogenous tracer within a subject indicative of a substance administered to the subject.

113. The method of claim 112, wherein the active substance and the exogenous tracer are simultaneously administered to the subject.

114. The method of claim 112, wherein the active substance and the exogenous tracer are each contained within a composition administered to the subject.

115. The method of claim 112, wherein the substance is a drug.

116-122. (canceled)

123. The method of claim 121, wherein the indicator comprises particles.

124. (canceled)

125. The method of claim 112, wherein the composition is administered orally to the subject.

126. The method of claim 112, wherein the composition is administered parenterally to the subject.

127. A kit, comprising;
a composition comprising a tracer and a carrier containing the tracer; and
a skin delivery device comprising an indicator responsive to the tracer.

128. (canceled)

129. The kit of claim 127, wherein the indicator comprises particles.

130. (canceled)

131. The kit of claim 127, wherein the skin delivery device is a skin patch.

132. The kit of claim 127, further comprising instructions for use of the composition and/or the skin delivery device.

133. A kit, comprising;
a composition comprising a tracer and a substance; and
a skin delivery device comprising an indicator responsive to the tracer.

134. The kit of claim 133, further comprising a carrier containing the tracer.

135. The kit of claim 134, wherein the carrier comprises the substance.

136. The kit of claim 133, wherein the substance is a drug.

137-140. (canceled)

141. A kit, comprising:
a tracer;
a substance; and
instructions for administering the tracer and the substance to a subject, and determining the tracer within the subject.

142. The kit of claim 141, wherein the substance is a drug.

143. (canceled)

144. The kit of claim 141, wherein the indicator comprises particles.

145-146. (canceled)

147. The kit of claim 146, wherein the indicator comprises particles.

148. A method, comprising:
offering, to a subject, an ingestible composition comprising a tracer and an ingestible substance, and a device comprising an indicator able to change a visual property upon exposure to the tracer.

149. The method of claim 148, wherein the substance is a drug.

150. (canceled)

151. The method of claim 148, wherein the composition is a liquid containing the tracer and the substance.

152. The method of claim 148, wherein the composition is a solid.

153. The method of claim 148, wherein the tracer and the substance are chemically unbonded to each other.

154. The method of claim 148, wherein the tracer and the substance are chemically bonded to each other.

155-156. (canceled)

157. The method of claim 148, wherein the indicator comprises particles.

158. (canceled)
